(19) United States
(12) Patent Application Publication
(10) Pub. No.: US 2026/0167595 A1
(43) Pub. Date: Jun. 18, 2026

(54) PRODUCTS AND METHODS RELATED TO THE DISTILLATION OF MOLECULES FROM AEROSOLS

(71) Applicant: Natural Extraction Systems, LLC, Boulder, CO (US)

(72) Inventor: C. Russell Thomas, Boulder, CO (US)

(21) Appl. No.: 19/369,841

(22) Filed: Oct. 27, 2025

Related U.S. Application Data
(63) Continuation-in-part of application No. 18/553,175, now Pat. No. 12,453,930.
(63) Continuation of application No. 19/117,556.
(60) Provisional application No. 63/170,306, filed on Apr. 2, 2021.
(60) Provisional application No. 63/173,195, filed on Apr. 9, 2021.
(60) Provisional application No. 63/250,861, filed on Sep. 30, 2021.
(60) Provisional application No. 63/250,916, filed on Sep. 30, 2021.
(60) Provisional application No. 63/412,426, filed on Oct. 1, 2022.
(60) Provisional application No. 63/412,427, filed on Oct. 1, 2022.

(51) Int. Cl. C07C 47/58

(52) U.S. Cl. CPC C07C 47/58

(57) ABSTRACT

Various aspects of this disclosure relate to the discovery that converting a solid or a liquid into an aerosol allows the distillation of molecules from the aerosol in seconds at temperatures that are significantly less than the boiling points of the molecules. Various aspects of this disclosure relate to the discovery that mute plants can be extracted at both ambient temperatures and temperatures significantly less than the boiling points of the volatile molecules of the mute plants by capturing the volatile molecules in a solvent such as ethanol.

### CROSS-REFERENCE TO RELATED APPLICATIONS

This patent application is a continuation of U.S. patent application Ser. No. 19/117,556, filed Apr. 1, 2025, which is the U.S. National Phase under 35 U.S.C. § 371 of International Application No. PCT/US2023/034314, which claims priority to U.S. Provisional Patent Application No. 63/412,426, filed Oct. 1, 2022, and U.S. Provisional Patent Application No. 63/412,427, filed Oct. 1, 2022, each of which is incorporated by reference in its entirety.

This patent application also claims priority to U.S. patent application Ser. No. 18/553,175, which granted as U.S. Pat. No. 12,453,930, and which is the U.S. National Phase under 35 U.S.C. § 371 of International Application No. PCT/US2022/023159, which claims priority to U.S. Provisional Patent Application No. 63/170,306, filed Apr. 2, 2021; U.S. Provisional Patent Application No. 63/173,195, filed Apr. 9, 2021; U.S. Provisional Patent Application No. 63/250,861, filed Sep. 30, 2021; and U.S. Provisional Patent Application No. 63/250,916, filed Sep. 30, 2021, each of which is incorporated by reference in its entirety.

### BACKGROUND

Natural product extraction is a mature technical field. An oligopoly of a few major flavor and fragrance companies achieved economies of scale and global footprints. Their products became commoditized and fungible, which forced cost-cutting and tight profit margins.

Many “current good manufacturing practices” utilize chemicals now known to present health risks and labor practices that draw an uncomfortable line between acceptable and exploitative. Regulators and customers increasingly pressure the ingredients industries, and newsworthy publications suggest that new regulations may inadvertently end the economically-viable production of several key ingredients used in everyday consumer packaged goods.

A new extraction technology that could increase profit margins without reliance upon disfavored solvents could allow flavor and fragrance companies to absorb the cost of additional regulation and save the global supply of ingredients that currently require manufacturing methods that may not remain tenable in the years to come.

### SUMMARY

Various aspects of this disclosure relate to the discovery that converting a solid or a liquid into an aerosol allows the distillation of molecules from the aerosol in seconds at temperatures that are significantly less than the boiling points of the molecules. This discovery extends beyond aerosols to many compositions that have large surface-area-to-volume ratios.

Various aspects of this disclosure relate to the discovery that mute plants, which were historically resistant to extraction and include, for example, lily of the valley, can be extracted at both ambient temperatures and temperatures significantly less than the boiling points of the volatile molecules of the mute plants by capturing the volatile molecules in a solvent such as ethanol. Such extractions are commercially viable, for example, by recirculating a gas through a first chamber that contains biomass of the mute plant and a second chamber that contains the solvent. This discovery extends beyond mute plants to other compositions that comprise volatile molecules.

### DETAILED DESCRIPTION

Various aspects of this disclosure relate to a method to separate a molecule from an impurity, comprising: providing a composition comprising the molecule and the impurity, wherein the molecule is present in the composition in a solid phase or a liquid phase, and the impurity is present in the composition in a solid phase or a liquid phase; converting the molecule into a vaporized molecule in a gas phase, wherein the gas phase has a pressure and a temperature, the molecule has a boiling point at the pressure and a vapor pressure at the temperature, the pressure of the gas phase is greater than the vapor pressure of the molecule, the boiling point of the molecule is greater than the temperature of the gas phase, and either the impurity lacks a vapor pressure or the impurity has a vapor pressure at the temperature that is less than the vapor pressure of the molecule at the temperature; separating the vaporized molecule from the impurity; and condensing the vaporized molecule into a condensed molecule. The precise nature of a distillation apparatus configured to perform a method of this disclosure is not limiting, and the methods of this disclosure may be performed, for example, in a system described in European Patent No. 3,283,606 B1.

In this disclosure, the term “boiling point” includes both the conventional definition of the term, and, when a molecule of this disclosure lacks a boiling point and has a sublimation point, then the term “boiling point” encompasses the term sublimation point in reference to such a molecule. Caffeine sublimes instead of boiling, for example, and, in this disclosure, the “boiling point” of caffeine refers to the sublimation point of caffeine.

In some embodiments, solid or liquid particles of a composition are introduced into a chamber, passageway, vessel, or tube that contains a moving sweep gas. The bombardment of the sweep gas aerosolizes the composition and transports it through the chamber, passageway, vessel or tube. The aerosolized composition remains in contact with the sweep gas for a period of time. Molecules evaporate from the composition to form a vapor. A method is provided to separate the evaporated vapor from the non-evaporated components of the composition, for example, such as by passing the aerosol through a cyclone. A method is then provided to separate the vapor from the sweep gas. The sweep gas containing the vaporized molecules may be passed through a spray or curtain of collection solvent or bubbled through a tank of collection solvent to condense the vaporized molecules into condensed molecules. The collection solvent may contain compounds that attract and absorb the vaporized molecules, thereby capturing a substantial portion of the vaporized molecules from the sweep gas and holding the condensed molecules in the collection solvent. A collection solvent may comprise ethanol, a mixture of ethanol and water, or other solvents or mixtures of solvents. The collection solvent may be advantageously selected from solvents or a mixture of solvents that the evaporated molecules are dissolvable or miscible within, for example, to facilitate pumping of the condensed molecules in the collection solvent, but such features do not limit this disclosure. Collection solvents are further described in European Patent No. 3,283,606 B1, which is incorporated its entirety by reference. Over time, more and more evaporated molecules are transferred from the composition to the collection solvent. Once the collection solvent has captured a sufficient quantity of the vaporized molecules, the collection solvent containing the condensed molecules may be removed from the extraction machine, either manually or automatically, and fresh collection solvent may be introduced back into the machine either manually or automatically. The removal and introduction of collection solvent may be performed in incremental batches or continuously.

While the inventor has scaled the preceding embodiment for commercial applications, numerous other methods to apply the concepts of this disclosure to the extractions of various plants may be preferable, for example, when an extract contains toxins or irritants such as bourgeonal of lily of the valley or capsaicin of chili peppers. In some embodiments, for example, a recirculating loop of sweep gas continuously passes through the composition. The composition may be held within a container, basket, mesh, or chamber such that the sweep gas is able to contact a substantial portion of the surface area of the composition, or, alternatively, the composition may be suspended in a fluidized bed. Molecules are evaporated from the composition to form a vapor. The sweep gas containing the vaporized molecules is passed through a spray or curtain of collection solvent or bubbled through a tank of collection solvent to condense the vaporized molecules into condensed molecules. The sweep gas that has passed through the collection solvent is recirculated through the composition. Over time, more and more molecules are transferred from the composition into the collection solvent. Once the collection solvent has captured a sufficient quantity of the molecules, the collection solvent containing the condensed molecules may be removed from the extraction machine, either manually or automatically, and fresh collection solvent may be introduced back into the machine either manually or automatically. The removal and introduction of collection solvent may be performed in incremental batches or continuously. In some embodiments, the sweep gas is not recirculated, but instead flows continuously through the composition.

In some embodiments, solid or liquid particles of a composition are introduced into a chamber, passageway, vessel, or tube that contains a moving sweep gas. The composition is bombarded by the sweep gas. Molecules evaporate from the composition to form a vapor. A method may be provided to separate the evaporated vapor from the non-evaporated components of the composition for example, such as by passing the aerosol through a cyclone. A method is then provided to separate the vapor from the sweep gas. The sweep gas containing the vaporized molecules may be passed through a spray or curtain of collection solvent or bubbled through a tank of collection solvent to condense the vaporized molecules into condensed molecules. Over time, more and more vaporized molecules are transferred from the composition into the collection solvent. Once the collection solvent has captured a sufficient quantity of the vaporized molecules, the collection solvent containing the condensed molecules may be removed from the extraction machine, either manually or automatically, and fresh collection solvent may be introduced back into the machine either manually or automatically. The removal and introduction of collection solvent may be performed in incremental batches or continuously.

“Comprising” refers to an open set, for example, such that a method comprising a number of disclosed steps can also comprise additional undisclosed steps.

In some embodiments, the method comprises bombarding the composition with at least 10 sextillion molecules of a sweep gas per gram of the composition. In some specific embodiments, the method comprises bombarding the composition with at least 10 sextillion molecules of a sweep gas per gram of the composition per second.

In some embodiments, the method comprises bombarding the composition with at least 1 liter of a sweep gas per gram of the composition. In some specific embodiments, the method comprises bombarding the composition with at least 1 liter of a sweep gas per gram of the composition per second.

In some embodiments, the method comprises bombarding the composition with a sweep gas at a force of at least 10 millinewtons per gram of the composition.

In some specific embodiments, the method comprises bombarding the composition with a sweep gas with an impulse of at least 10 millinewton-seconds per gram of the composition.

In some embodiments, the method comprises bombarding the composition with a sweep gas that has a kinetic energy of at least 1 millijoule per gram of the composition.

In some embodiments, the method comprises bombarding the composition with a sweep gas that has a velocity of at least 100 millimeters per second.

In some embodiments, the method comprises bombarding the composition with a sweep gas for no greater than 60 seconds. In some specific embodiments, the method comprises bombarding the composition with a sweep gas for at least 100 milliseconds and no greater than 10 seconds.

In some embodiments, the composition off-gasses the vaporized molecule at a vaporization rate; the vaporized molecule recondenses onto the composition at a condensation rate; converting the molecule into the vaporized molecule occurs at a mass transfer rate, which is equal to the vaporization rate minus the condensation rate; and the bombarding increases the mass transfer rate.

In some embodiments, the bombarding decreases the condensation rate.

In some embodiments, the bombarding increases the vaporization rate. In some specific embodiments, the bombarding increases the vaporization rate to at least 5 micrograms of the molecule per gram of the composition per second.

In some embodiments, the bombarding increases the mass transfer rate to at least 5 micrograms of the molecule per gram of the composition per second.

In some embodiments, the vaporized molecule has a partial pressure at the surface of the composition; and the bombarding decreases the partial pressure of the vaporized molecule at the surface of the composition.

In some embodiments, the vaporized molecule has a partial pressure at the surface of the composition; the vaporized molecule recondenses onto the composition at a condensation rate; decreasing the partial pressure of the vaporized molecule at the surface of the composition decreases the condensation rate; and the bombarding both decreases the partial pressure of the vaporized molecule at the surface of the composition and decreases the condensation rate.

In some embodiments, the vaporized molecule has a partial pressure at the surface of the composition; the composition off-gasses the vaporized molecule at a vaporization rate; the vaporized molecule recondenses onto the composition at a condensation rate; converting the molecule into the vaporized molecule occurs at a mass transfer rate, which is equal to the vaporization rate minus the condensation rate; decreasing the partial pressure of the vaporized molecule at the surface of the composition increases the mass transfer rate; and the bombarding both decreases the partial pressure of the vaporized molecule at the surface of the composition and increases the mass transfer rate.

In some embodiments, the vaporized molecule has a partial pressure in the gas phase; the partial pressure of the vaporized molecule has concentration gradients in the gas phase; the concentration gradients have magnitudes; and the bombarding decreases the magnitudes of the concentration gradients.

In some embodiments, the vaporized molecule has a partial pressure in the gas phase; the partial pressure of the vaporized molecule in the gas phase inversely correlates with distance from the composition; and the bombarding decreases the inverse correlation.

In some embodiments, the vaporized molecule has a partial pressure in the gas phase; the partial pressure of the vaporized molecule in the gas phase inversely correlates with distance from the composition; the inverse correlation has a magnitude; and the bombarding decreases the magnitude of the inverse correlation.

In some embodiments, the vaporized molecule has a partial pressure in the gas phase; the partial pressure of the vaporized molecule in the gas phase inversely correlates with distance from the composition; the inverse correlation has a correlation coefficient of at least −1 and less than 0, wherein −1 is complete inverse correlation and 0 is no correlation; the correlation coefficient has an absolute value; and the bombarding decreases the absolute value of the correlation coefficient.

In some embodiments, the bombarding performs work on the vaporized molecule. In some specific embodiments, the bombarding performs work on the vaporized molecule that translates the vaporized molecule in three-dimensional space.

In some embodiments, the bombarding performs work on the vaporized molecule that translates the vaporized molecule by at least 1 centimeter. In some specific embodiments, the bombarding performs work on the vaporized molecule that translates the vaporized molecule by at least 1 meter.

In some embodiments, the bombarding transfers kinetic energy to the vaporized molecule. In some specific embodiments, the bombarding transfers at least 10 microjoules of kinetic energy to the vaporized molecule per gram of the vaporized molecule.

In some embodiments, the bombarding accelerates the vaporized molecule. In some specific embodiments, the bombarding accelerates the vaporized molecule to an average velocity of at least 10 millimeters per second. In some very specific embodiments, the bombarding accelerates the vaporized molecule to an average velocity of at least 100 millimeters per second.

In some embodiments, the bombarding increases the vapor pressure of the molecule. In some specific embodiments, the composition off-gasses the vaporized molecule at a vaporization rate; increasing the vapor pressure of the molecule increases the vaporization rate; and the bombarding both increases the vapor pressure of the molecule and increases the vaporization rate. In some very specific embodiments, the bombarding increases the vaporization rate to at least 5 micrograms of the molecule per gram of the composition per second.

In some embodiments, the composition off-gasses the vaporized molecule at a vaporization rate; the vaporized molecule recondenses onto the composition at a condensation rate; converting the molecule into the vaporized molecule occurs at a mass transfer rate, which is equal to the vaporization rate minus the condensation rate; increasing the vapor pressure of the molecule increases the mass transfer rate; and the bombarding both increases the vapor pressure of the molecule and increases the mass transfer rate. In some specific embodiments, the bombarding increases the mass transfer rate to at least 5 micrograms of the molecule per gram of the composition per second.

In some embodiments, the composition has thermal energy; the bombarding increases the thermal energy of the composition; and increasing the thermal energy of the composition increases the vapor pressure of the molecule.

In some embodiments, increasing the vapor pressure of the molecule comprises sensible heat transfer from the gas phase to the composition; the sensible heat transfer has a rate; and the bombarding increases the rate of the sensible heat transfer. In some specific embodiments, the sensible heat transfer from the gas phase to the composition is completed in less than 60 seconds.

In some embodiments, the bombarding results in a rate of sensible heat transfer from the sweep gas to the composition of no greater than 1 kilojoule per gram of the composition per second. In some specific embodiments, the bombarding results in a rate of sensible heat transfer from the sweep gas to the composition of no greater than 100 joules per gram of the composition per second. In some very specific embodiments, the bombarding results in a rate of sensible heat transfer from the sweep gas to the composition of no greater than 20 joules per gram of the composition per second.

In some embodiments, converting the molecule into the vaporized molecule comprises latent heat transfer between the composition and the gas phase; the latent heat transfer has a rate; and the bombarding increases the rate of the latent heat transfer. In some specific embodiments, the latent heat transfer between the composition and the gas phase is completed in less than 60 seconds.

In some embodiments, the bombarding causes latent heat transfer between the sweep gas and the composition of no greater than 1 kilojoule per gram of the composition per second. In some specific embodiments, the bombarding causes latent heat transfer between the sweep gas and the composition of no greater than 500 joules per gram of the composition per second.

In some embodiments, the bombarding suspends at least 75 percent of the composition in the gas phase. In some specific embodiments, the bombarding suspends at least 98 percent of the composition in the gas phase.

In some embodiments, the bombarding performs work on the composition. In some specific embodiments, the bombarding performs work on the composition that translates at least 90 percent of the composition. In some very specific embodiments, the bombarding performs work on the composition that translates at least 90 percent of the composition by at least 1 meter.

In some embodiments, the bombarding transfers kinetic energy to the composition. In some specific embodiments, the bombarding transfers at least 10 microjoules of kinetic energy to the composition per gram of the composition.

In some embodiments, the bombarding accelerates the composition. In some specific embodiments, the bombarding accelerates at least 90 percent of the composition to an average velocity that is greater than 100 millimeters per second.

In some embodiments, the method comprises sensible heat transfer from the gas phase to the composition, wherein the sensible heat transfer has a rate, and the bombarding increases the rate of the sensible heat transfer.

In some embodiments, the composition has a temperature that is less than the temperature of the gas phase when the composition is provided; the method comprises heating the composition; and the bombarding heats the composition. In some specific embodiments, the composition has a temperature of no greater than 100 degrees Celsius when the composition is provided; the method comprises heating the composition to a temperature greater than 100 degrees Celsius; and the bombarding heats the composition. In some very specific embodiments, the composition has a temperature of at least 15 degrees Celsius and no greater than 100 degrees Celsius when the composition is provided; the method comprises heating the composition to a temperature greater than 100 degrees Celsius; and the bombarding heats the composition.

In some embodiments, the bombarding performs work that separates the vaporized molecule from the impurity.

In some embodiments, the bombarding propels the vaporized molecule through a cyclone or centrifugal separator that separates the vaporized molecule from the impurity.

In some embodiments, the bombarding propels the impurity through a cyclone or centrifugal separator that separates the vaporized molecule from the impurity.

In some embodiments, the bombarding propels the vaporized molecule through a filter that separates the vaporized molecule from the impurity.

In some embodiments, the method comprises providing a system, wherein converting the molecule into the vaporized molecule is performed in a first chamber of the system; condensing the vaporized molecule into the condensed molecule is performed in a second chamber of the system; and the bombarding propels the vaporized molecule from the first chamber of the system to the second chamber of the system.

In some embodiments, the bombarding propels the vaporized molecule to a compressor that condenses the vaporized molecule into the condensed molecule.

In some embodiments, the bombarding propels the vaporized molecule to a heat sink that condenses the vaporized molecule into the condensed molecule.

In some embodiments, the sweep gas comprises one or more of molecular nitrogen, molecular oxygen, carbon dioxide, argon, neon, water vapor, and ethanol vapor. In some specific embodiments, the sweep gas comprises one or more of molecular nitrogen, molecular oxygen, carbon dioxide, argon, neon, water vapor, and ethanol vapor at a combined concentration of at least 50 percent by mass. In some very specific embodiments, the sweep gas consists of one or more of molecular nitrogen, molecular oxygen, carbon dioxide, argon, neon, water vapor, and ethanol vapor.

“Consists of” refers to a closed set, for example, such that a sweep gas that consists of one or more disclosed molecules cannot also comprise undisclosed molecules. The skilled person nevertheless understands that impurities might be present at detectable, trace concentrations in the real-world practice of the inventions of this disclosure, and the term “consists of” allows for the presence of undisclosed impurities in a sweep gas that “consists of” one or more disclosed molecules when both (i) the undisclosed impurities are present at lower concentrations than the disclosed molecules and (ii) the undisclosed impurities do not affect the practice of the methods of the disclosure.

In some embodiments, the sweep gas comprises molecular nitrogen at a concentration of at least 50 percent by mass.

In some embodiments, the sweep gas comprises steam at a concentration of at least 50 percent by mass. In some specific embodiments, the sweep gas consists of steam.

In some embodiments, the sweep gas has a Reynolds number of at least 1 during the bombarding.

In some embodiments, the sweep gas has a Reynolds number of no greater than 100,000 during the bombarding.

In some embodiments, the composition has a drag coefficient of at least 0.5 when the composition is bombarded with the sweep gas.

In some embodiments, the method comprises processing a starting composition to increase its surface-area-to-volume ratio, wherein providing the composition comprises the processing. In some specific embodiments, the composition off-gasses the vaporized molecule at a vaporization rate; a greater surface-area-to-volume ratio correlates with a greater vaporization rate; providing the composition comprises preparing the composition from a starting composition; the starting composition has a surface-area-to-volume ratio that is less than the surface-area-to-volume ratio of the composition; and the processing comprises one or both of increasing the surface-area-to-volume ratio of the starting composition and selecting a portion of the starting composition that has a greater surface-area-to-volume ratio than the rest of the starting composition.

In some embodiments, providing the composition comprises one or both of grinding a starting composition and separating the starting composition by size.

In some embodiments, providing the composition comprises selecting particles of a starting composition that have a particle size of no greater than 5 millimeters.

“Particle size” refers to the longest linear distance that connects one point of a particle of the composition to another point of the particle in three-dimensional Euclidean space.

In some embodiments, providing the composition comprises grinding a starting composition to an average particle size that is no greater than 5 millimeters.

In some embodiments, the composition off-gasses the vaporized molecule at a vaporization rate, and the surface-area-to-volume ratio of the composition supports a vaporization rate of at least 5 micrograms of the molecule per gram of the composition per second at the temperature and the pressure of the gas phase.

In some embodiments, the composition off-gasses the vaporized molecule at a vaporization rate; the vaporized molecule recondenses onto the composition at a condensation rate; converting the molecule into the vaporized molecule occurs at a mass transfer rate, which is equal to the vaporization rate minus the condensation rate; and the surface-area-to-volume ratio of the composition supports a mass transfer rate of at least 5 micrograms of the molecule per gram of the composition per second at the temperature and the pressure of the gas phase.

In some embodiments, the composition has a surface-area-to-volume ratio that is greater than 500 per meter. In some specific embodiments, the composition has a surface-area-to-volume ratio of at least 2400 per meter.

In some embodiments, the composition comprises flowers, flower petals, or partially-processed flowers, and the composition has a surface-area-to-volume ratio that is less than 500 per meter. In some specific embodiments, the flowers or petals are mostly whole.

In some embodiments, the composition has an average terminal velocity of no greater than 5 meters per second in still, dry air at 1 atmosphere of pressure.

In some embodiments, providing the composition comprises selecting a portion of a starting composition that has a terminal velocity of no greater than 5 meters per second in still, dry air at 1 atmosphere of pressure.

In some embodiments, the method comprises suspending at least 75 percent of the composition in the gas phase. In some specific embodiments, the method comprises suspending at least 98 percent of the composition in the gas phase.

In some embodiments, the composition is not suspended in the gas phase when the molecule is converted into the vaporized molecule.

In some embodiments, the impurity is a monosaccharide, disaccharide, or polysaccharide. In some specific embodiments, the impurity is cellulose I.

In some embodiments, the impurity is an amino acid, polypeptide, or protein.

In some embodiments, the impurity is a nucleobase, nucleoside, nucleotide, or nucleic acid.

In some embodiments, the impurity is a triglyceride.

In some embodiments, the impurity is chlorophyll.

In some embodiments, the impurity is sodium ion, potassium ion, calcium ion, iron(II), iron(III), magnesium ion, or phosphate.

In some embodiments, the composition comprises biological cells; the biological cells have interiors, exteriors, and cell membranes that separate the interiors from the exteriors; and the method comprises generating sufficient pressure within the biological cells to rupture at least 10 percent of the cell membranes. Rupturing cell membranes improves extraction by facilitating fluid communication between the interiors and the gas phase.

In some embodiments, the composition comprises biological cells; the biological cells have interiors, exteriors, and cell membranes that separate the interiors from the exteriors; the method comprises vaporizing an accessory molecule within the biological cells; and vaporizing the accessory molecule generates sufficient pressure within the biological cells to rupture at least 10 percent of the cell membranes.

In some embodiments, the composition comprises biological cells; the biological cells have interiors, exteriors, and cell membranes that separate the interiors from the exteriors; and the method comprises vaporizing an accessory molecule within the biological cells at a rate sufficient to generate pressure within the biological cells that ruptures at least 10 percent of the cell membranes.

In some embodiments, the method comprises vaporizing the accessory molecule and rupturing the cell membranes in a total time of no greater than 60 seconds.

In some embodiments, the composition comprises the accessory molecule at a concentration of at least 1000 parts per million by mass.

In some embodiments, the composition comprises the accessory molecule at a concentration of no greater than 20 percent by mass.

In some embodiments, the accessory molecule is water.

In some embodiments, the method comprises generating sufficient pressure within the biological cells to rupture at least 75 percent of the cell membranes.

In some embodiments, the composition off-gasses the vaporized molecule at a vaporization rate, and the rupturing increases the vaporization rate.

In some embodiments, rupturing increases the vaporization rate to at least 5 micrograms of the molecule per gram of the composition per second.

In some embodiments, the composition off-gasses the vaporized molecule at a vaporization rate; the vaporized molecule recondenses onto the composition at a condensation rate; converting the molecule into the vaporized molecule occurs at a mass transfer rate, which is equal to the vaporization rate minus the condensation rate; and the rupturing increases the mass transfer rate. In some specific embodiments, the rupturing increases the mass transfer rate to at least 5 micrograms of the molecule per gram of the composition per second.

In some embodiments, the composition comprises the molecule at a concentration of at least 10 parts per million and no greater than 1 percent by mass.

In some embodiments, the boiling point of the molecule at the pressure of the gas phase is at least 10 percent greater than the boiling point of water in Celsius at the pressure of the gas phase.

In some embodiments, the boiling point of the molecule at the pressure of the gas phase is at least 5 percent greater than the temperature of the gas phase in Celsius. In some specific embodiments, the boiling point of the molecule at the pressure of the gas phase is at least 20 percent greater than the temperature of the gas phase in Celsius. In some very specific embodiments, the boiling point of the molecule at the pressure of the gas phase is at least 40 percent greater than the temperature of the gas phase in Celsius.

In some embodiments, the boiling point of the molecule at the pressure of the gas phase is greater than 100 degrees Celsius. In some specific embodiments, the boiling point of the molecule at the pressure of the gas phase is greater than 230 degrees Celsius. In some very specific embodiments, the boiling point of the molecule at the pressure of the gas phase is greater than 250 degrees Celsius.

In some embodiments, the vapor pressure of the molecule at the temperature of the gas phase is less than the vapor pressure of water at the temperature of the gas phase. In some specific embodiments, the vapor pressure of the molecule at the temperature of the gas phase is no greater than 50 percent of the vapor pressure of water at the temperature of the gas phase.

In some embodiments, the vapor pressure of the molecule at the temperature of the gas phase is no greater than 90 percent of the pressure of the gas phase.

In some embodiments, the vapor pressure of the molecule at the temperature of the gas phase is at least 1 percent of the pressure of the gas phase.

In some embodiments, the temperature of the gas phase is greater than the boiling point of water at the pressure of the gas phase.

In some embodiments, the temperature of the gas phase is less than 250 degrees Celsius.

In some embodiments, the pressure of the gas phase is at least 0.5 atmospheres and no greater than 2 atmospheres.

In some embodiments, the method comprises converting at least 10 percent of the molecule into the condensed molecule by mole. In some specific embodiments, the method comprises converting at least 60 percent of the molecule into the condensed molecule by mole.

In some embodiments, the composition comprises a starting ratio of the molecule to the impurity by mass; condensing the vaporized molecule into a condensed molecule results in a condensed phase that comprises an ending ratio of the molecule to the impurity by mass; and the ending ratio is at least 5 times greater than the starting ratio. In some specific embodiments, the ending ratio is at least 50 times greater than the starting ratio. In some very specific embodiments, the ending ratio is at least 500 times greater than the starting ratio.

In some embodiments, condensing the vaporized molecule into a condensed molecule comprises increasing the pressure of the gas phase, reducing the temperature of the gas phase, or both increasing the pressure of the gas phase and reducing the temperature of the gas phase.

In some embodiments, condensing the vaporized molecule into the condensed molecule comprises contacting the vaporized molecule with a heat sink.

In some embodiments, the method comprises converting the molecule into the vaporized molecule in a system that contains the gas phase, wherein the system is configured to inhibit the gas phase from escaping the system.

In some embodiments, the method comprises providing a system, wherein converting the molecule into the vaporized molecule is performed in a first chamber of the system and condensing the vaporized molecule into the condensed molecule is performed in a second chamber of the system.

In some embodiments, the system allows passage of the vaporized molecule from the first chamber to the second chamber.

In some embodiments, the system allows passage of the gas phase from the first chamber to the second chamber.

In some embodiments, the system inhibits passage of the impurity from the first chamber to the second chamber.

In some embodiments, the system inhibits passage of the composition from the first chamber to the second chamber.

In some embodiments, the system inhibits passage of solids from the first chamber to the second chamber.

In some embodiments, the system inhibits passage of liquids from the first chamber to the second chamber.

In some embodiments, the system allows passage of gases from the second chamber to the first chamber.

In some embodiments, the method comprises condensing the vaporized molecule into the condensed molecule from a first portion of the composition in the second chamber and concurrently converting the molecule into the vaporized molecule from a subsequent portion of the composition in the first chamber.

In some embodiments, the method comprises feeding the composition into the first chamber of the system at a feed rate, which is the amount of the molecule that is fed into the first chamber per unit time; converting the molecule into the vaporized molecule at a mass transfer rate, which is the amount of the molecule that the composition off-gases minus the amount of the vaporized molecule that recondenses onto the composition per unit time; and condensing the vaporized molecule into the condensed molecule at a collection rate, which is the amount of the vaporized molecule that is condensed into the condensed molecule per unit time, wherein the method is performed such that the collection rate is at least 50 percent and no greater than 100 percent of the mass transfer rate over a period of time; the mass transfer rate is at least 50 percent and no greater than 100 percent of the feed rate over a concurrent period of time; and the period of time is chronologically identical to the concurrent period of time. In some specific embodiments, the period of time and the concurrent period of time are the same 10 second period. In some specific embodiments, the period of time and the concurrent period of time are the same 5 second period. In some specific embodiments, the period of time and the concurrent period of time are the same 1 second period.

In some embodiments, a vacuum pump applies a partial vacuum to the composition. In some specific embodiments, a vacuum pump applies a partial vacuum to the composition during the bombarding of the composition with the sweep gas.

In some embodiments, the method comprises drawing the vaporized molecule through the vacuum pump. In some specific embodiments, the method comprises drawing the vaporized molecule and the sweep gas through the vacuum pump.

In some embodiments, the molecule is not water.

In some embodiments, the molecule is a furan.

In some embodiments, the molecule is furyl-hydroxymethyl ketone (CAS: 17678-19-2); 2-methyl-benzofuran (CAS: 4265-25-2); 2-(2-furanylmethyl)-5-methyl-furan (CAS 13678-51-8); or 2,5-furandicarboxaldehyde (CAS: 823-82-5).

In some embodiments, the molecule is an alcohol.

In some embodiments, the molecule is nonan-1-ol (CAS: 143-08-8); decan-1-ol (CAS: 112-53-8); dodec-1-ol (CAS: 112-53-8); tetradecane-1-ol (CAS: 112-72-1); hexadecane-1-ol (CAS: 36653-82-4); or octadecane-1-ol (CAS: 112-92-5).

In some embodiments, the molecule is a fatty alcohol that comprises at least 9 carbon atoms.

In some embodiments, the molecule is nonen-3-ol (CAS: 21964-44-3).

In some embodiments, the molecule is an unsaturated alcohol.

In some embodiments, the molecule is 2-decen-1-ol (CAS: 22104-80-9).

In some embodiments, the molecule is an aromatic alcohol.

In some embodiments, the molecule is an aldehyde.

In some embodiments, the molecule is an unsaturated aldehyde.

In some embodiments, the molecule is a substituted aldehyde.

In some embodiments, the molecule is a unsubstituted aldehyde.

In some embodiments, the molecule is tetradecanal (CAS: 124-25-4); (Z)-2-decenal (CAS: 2497-25-8); or (E,E)-2,4-decadienal (CAS: 25152-84-5).

In some embodiments, the molecule is a carboxylic acid.

In some embodiments, the molecule is a low molecular weight volatile acid.

In some embodiments, the molecule is a fatty acid.

In some embodiments, the molecule is an unsaturated fatty acid.

In some embodiments, the molecule is a saturated fatty acid.

In some embodiments, the molecule is octanoic acid (CAS: 124-07-2); nonanoic acid (CAS: 112-05-0); n-decanoic acid (CAS: 334-48-5); n-hexadecanoic acid (CAS: 57-10-3); heptadecanoic acid (CAS: 506-12-7); or octadecanoic acid (CAS: 57-11-4).

In some embodiments, the molecule is a fatty acid.

In some embodiments, the molecule is a substituted carboxylic acid.

In some embodiments, the molecule is 2-ethylhexanoic acid (CAS: 149-57-5).

In some embodiments, the molecule is an unsaturated carboxylic acid.

In some embodiments, the molecule is trans-2-undecenoic acid (CAS: 15790-94-0).

In some embodiments, the molecule is an aromatic acid.

In some embodiments, the molecule is benzoic acid (CAS: 65-85-0); or phthalic acid (CAS: 88-99-3).

In some embodiments, the molecule is an ester.

In some embodiments, the molecule is a branched ester.

In some embodiments, the molecule is a non-branched ester.

In some embodiments, the molecule is a primary, secondary, tertiary, substituted, or unsubstituted, ester.

In some embodiments, the molecule is a mono-ester.

In some embodiments, the molecule is a di-ester.

In some embodiments, the molecule is an ester of a saturated acid.

In some embodiments, the molecule is an ester of an unsaturated acid.

In some embodiments, the molecule is an ester of an aromatic acid.

In some embodiments, the molecule is methyl nonanoate (CAS: 1731-84-6); octyl butanoate (CAS: 110-39-4); isopropyl myristate (CAS: 110-27-0); ethyl nicotinate (CAS: 614-18-6); 3-hexenyl butanoate (CAS: 53398-84-8); diethyl butanedioate (CAS: 123-25-1); or diethyl itaconate (CAS: 2409-52-1).

In some embodiments, the molecule is a chemical derived from lignin.

In some embodiments, the molecule is a substituted phenol.

In some embodiments, the molecule is methyl salicylate (CAS: 119-36-8); eugenol (CAS 97-53-0); vanillin (CAS 121-33-5); (Z)-isoeugenol (CAS: 5932-68-3); ethyl anisate (CAS: 94-30-4); 4-methylguauacol (CAS: 93-51-6); or (E)-2,6-dimethoxy-4-(prop-1-en-1-yl)phenol (CAS: 20675-95-0).

In some embodiments, the molecule is a terpene.

In some embodiments, the molecule is 1,3,8-p-menthatriene, (CAS: 18368-95-1).

In some embodiments, the molecule is a monoterpene.

In some embodiments, the molecule is a sesquiterpene.

In some embodiments, the molecule is an oxide of a terpene.

In some embodiments, the molecule is an alkaloid.

In some embodiments, the molecule is a nitrogen-containing volatile compound.

In some embodiments, the molecule is caffeine (CAS: 58-08-2).

In some embodiments, the molecule is nicotine (CAS: 54-11-5).

In some embodiments, the molecule is 1H-pyrrole-2-carboxaldehyde (CAS: 1003-29-8).

In some embodiments, the molecule is a phytosterol.

In some embodiments, the molecule is a derivative of a phytosterol.

In some embodiments, the molecule is (4R,5R)-5-butyl-4-methyloxolan-2-one (CAS: 55013-32-6); (4S,5R)-5-butyl-4-methyloxolan-2-one (CAS: 39638-67-0); β-ionone (CAS: 8013-90-9); or 6-tetradecalactone (CAS: 2721-22-4).

In some embodiments, the molecule is an organosulfur compound.

In some embodiments, the molecule is bis(2-furfuryl)disulfide (CAS: 4437-20-1).

In some embodiments, the molecule is an alkane.

In some embodiments, the molecule is an alkene.

In some embodiments, the molecule is a hydrocarbon.

In some embodiments, the molecule is hydrocoumarin (CAS: 119-84-6).

In some embodiments, the molecule is coumarin (CAS: 91-64-5).

In some embodiments, the molecule is acetophenone (CAS: 98-86-2); alpha-bergamotol (CAS: 88034-74-6); alpha-bisabolol (CAS: 515-69-5); alpha-bisabolol oxide A (CAS: 22567-36-8); alpha-cadinol (CAS: 481-34-5); alpha-curcumene (CAS: 644-30-4); alpha-fenchene (CAS: 471-84-1); alpha-phellandrene (CAS: 99-83-2); alpha-pinene (CAS: 80-56-8); alpha-santalol (CAS: 115-71-9); alpha-terpinene (CAS: 99-86-5); alpha-terpineol (CAS: 98-55-5); alpha-terpinyl acetate (CAS: 80-26-2); alpha-thujene (CAS: 2867-05-2); alpha-thujone (CAS: 546-80-5); alpha-zingiberene (CAS: 495-60-3); azulene (CAS: 275-51-4); benzyl acetate (CAS: 140-11-4); benzyl benzoate (CAS: 120-51-4); bergamotene (CAS: 6895-56-3); beta-bisabolene (CAS: 495-61-4); beta-caryophyllene (CAS: 87-44-5); beta-damascenone (CAS: 23696-85-7); beta-eudesmol (CAS: 473-15-4); beta-farnesene (CAS: 77129-48-7); beta-phellandrene (CAS: 555-10-2); beta-pinene (CAS: 127-91-3); beta-santalol (CAS: 11031-45-1); beta-selinene (CAS: 17066-67-0); beta-sesquiphellandrene (CAS: 20307-83-9); beta-terpinene (CAS: 99-84-3); beta-terpinyl acetate (CAS: 10198-23-9); beta-thujene (CAS: 28634-89-1); beta-thujone (CAS: 1125-12-8); borneol (CAS: 464-45-9); bornyl acetate (CAS: 76-49-3); camphene (CAS: 79-92-5); camphor (CAS: 76-22-2); capsaicin (CAS: 404-86-4); carene (CAS: 13466-78-9); carvacrol (CAS: 499-75-2); carvone (CAS: 99-49-0); caryophyllene oxide (CAS: 1139-30-6); cedrene (CAS: 469-61-4); cedrol (CAS: 77-53-2); chamazulene (CAS: 529-05-5); chavicol (CAS: 501-92-8); cinnamaldehyde (CAS: 104-55-2); citral (CAS: 5392-40-5); citronellal (CAS: 106-23-0); citronellol (CAS: 106-22-9); citronellyl formate (CAS: 105-85-1); curzerene (CAS: 17910-09-7); cyclopentadecanolide (CAS: 106-02-5); decanal (CAS: 112-31-2); delta-guaiene (CAS: 3691-11-0); ethyl cinnamate (CAS: 103-36-6); eugenol (CAS: 97-53-0); farnesene (CAS: 502-61-4); farnesol (CAS: 4602-84-0); furanoeudesma-1,3-diene (CAS: 87605-93-4); furfural (CAS: 98-01-1); furfuryl acetate (CAS: 623-17-6); gamma-decalactone (CAS: 706-14-9); gamma-muurolene (CAS: 30021-74-0); gamma-nonalactone (CAS: 104-61-0); gamma-terpinene (CAS: 99-85-4); gamma-terpinyl acetate (CAS: 10235-63-9); geraniol (CAS: 106-24-1); geranyl acetate (CAS: 105-87-3); germacrene A (CAS: 28387-44-2); germacrene D (CAS: 37839-63-7); guaiacol (CAS: 90-05-1); heneicosane (CAS: 629-94-7); humulene (CAS: 6753-98-6); isoamyl benzoate (CAS: 94-46-2); kessane (CAS: 3321-66-2); limonene (CAS: 6876-12-6); linalool (CAS: 78-70-6); linalool oxide (CAS: 1365-19-1); linalyl acetate (CAS: 115-95-7); menthol (CAS: 89-78-1); menthone (CAS: 89-80-5); methyl cinnamate (CAS: 103-26-4); methyl eugenol (CAS: 93-15-2); methylpyrazine (CAS: 109-08-0); myrcene (CAS: 123-35-3); myristicin (CAS: 607-91-0); neral (CAS: 5392-40-5); nerol (CAS: 106-25-2); nerolidol (CAS: 7212-44-4); nookatone (CAS: 91416-23-8); nootkatin (CAS: 4431-03-2); nootkatol (CAS: 53643-07-5); nootkatone (CAS: 91416-23-8); ocimene (CAS: 7216-56-0); octanal (CAS: 124-13-0); para-cresol (CAS: 106-44-5); para-cymene (CAS: 99-87-6); patchouli alcohol (CAS: 5986-55-0); perillene (CAS: 539-52-6); phenylacetaldehyde (CAS: 122-78-1); phenylacetic acid (CAS: 103-82-2); phenylethyl alcohol (CAS: 60-12-8); phytol (CAS: 150-86-7); sabinene (CAS: 3387-41-5); safrole (CAS: 94-59-7); tau-muurolol (CAS: 19912-62-0); terpinen-4-ol (CAS: 562-74-3); terpinolene (CAS: 586-62-9); thymol (CAS: 89-83-8); valencene (CAS: 4630-07-3); vanillin (CAS: 121-33-5); zingerone (CAS: 122-48-5); zingiberenol (CAS: 58334-55-7); zingiberol (CAS: 6754-68-3); 1,8-cineole (CAS: 470-82-6); 1-phenylethyl acetate (CAS: 93-92-5); 2,6-dimethylpyrazine (CAS: 108-50-9); 2-furanmethanol (CAS: 98-00-0); 2-heptanol (CAS: 543-49-7); 2-heptanone (CAS: 110-43-0); 2-heptyl acetate (CAS: 5921-82-4); 2-methoxy-4-vinylphenol (CAS: 7786-61-0); 2-methyl-3-buten-2-ol (CAS: 115-18-4); 2-methylbutanoic acid (CAS: 116-53-0); 2-nonanone (CAS: 821-55-6); 2-pentanol (CAS: 6032-29-7); 2-pentyl acetate (CAS: 626-38-0); 2-phenylethyl alcohol (CAS: 60-12-8); 2-undecanone (CAS: 112-12-9); 3-methylbutanoic acid (CAS: 503-74-2); 3-phenylpropanoic acid (CAS: 501-52-0); 4-methylguaiacol (CAS: 93-51-6); 5-methylfurfural (CAS: 620-02-0); 6-gingerol (CAS: 23513-14-6); 6-methyl-5-hepten-2-one (CAS: 110-93-0); or 6-shogaol (CAS: 555-66-8).

In some embodiments, the molecule is phenylacetaldehyde oxime (CAS: 7028-48-0).

In some embodiments, the molecule is dihydrofarnesal (CAS: 32480-08-3).

In some embodiments, the composition is not cannabis, and the composition lacks any product that was derived from cannabis.

“Cannabis” refers to plants of the genus Cannabis and any portion of a plant of the genus Cannabis. Cannabis includes, for example, marijuana and industrial hemp.

“Any chemical species derived from cannabis” includes, for example chemical species that are extracted from cannabis and chemical species that are manufactured from cannabis such as by decarboxylating a cannabis extract comprising one or more cannabinoid carboxylic acids.

In some embodiments, the composition comprises biomass of a perennial plant.

In some embodiments, the composition comprises biomass of an annual plant.

In some embodiments, the composition comprises agave.

In some embodiments, the composition comprises mescal bagasse or tequila bagasse.

In some embodiments, the composition comprises sugarcane.

In some embodiments, the composition comprises sugarcane bagasse.

In some embodiments, the composition comprises sorghum bagasse.

In some embodiments, the composition comprises peat or smoked peat.

In some embodiments, the composition comprises malted grain.

In some embodiments, the composition comprises barley.

In some embodiments, the composition comprises or malted barley.

In some embodiments, the composition comprises corn.

In some embodiments, the composition comprises corn fibers, corn cobs, or corn bagasse.

In some embodiments, the composition comprises rice.

In some embodiments, the composition comprises fruit.

In some embodiments, the composition comprises citrus fruit.

In some embodiments, the composition comprises stone fruit.

In some embodiments, the composition comprises the wood of a stone fruit tree.

In some embodiments, the composition comprises aggregate fruit.

In some embodiments, the composition comprises berries.

In some embodiments, the composition comprises drupes.

In some embodiments, the composition comprises achenes.

In some embodiments, the composition comprises pineapple.

In some embodiments, the composition comprises tomato leaves.

In some embodiments, the composition comprises dried vegetables.

In some embodiments, the composition comprises dried peels.

In some embodiments, the composition comprises dried citrus peels.

In some embodiments, the composition comprises a species of ylang ylang.

In some embodiments, the composition comprises spices.

In some embodiments, the composition comprises herbs.

In some embodiments, the composition comprises flowers.

In some embodiments, the composition comprises seeds.

In some embodiments, the composition comprises stems.

In some embodiments, the composition comprises roots.

In some embodiments, the composition comprises leaves.

In some embodiments, the composition comprises rhizomes.

In some embodiments, the composition comprises a fungus.

In some embodiments, the composition comprises yeast.

In some embodiments, the composition comprises dried yeast.

In some embodiments, the composition comprises mushrooms.

In some embodiments, the composition comprises algae.

In some embodiments, the composition comprises bacteria.

In some embodiments, the composition is comprised primarily of dried bacteria.

In some embodiments, the composition comprises wood.

In some embodiments, the composition comprises sawdust.

In some embodiments, the composition comprises heartwood.

In some embodiments, the composition comprises Amburana wood.

In some embodiments, the composition comprises Amburana cearensis.

In some embodiments, the composition comprises a species of sandalwood (Santalum).

In some embodiments, the composition comprises Palo Santo wood (Bursera graveolens).

In some embodiments, the composition comprises a species of oak (Quercus).

In some embodiments, the composition comprises American oak (Quercus alba).

In some embodiments, the composition comprises French oak (Quercus robur).

In some embodiments, the composition comprises English oak (Quercus petraea).

In some embodiments, the composition comprises Hungarian oak (Quercus frainetto).

In some embodiments, the composition comprises mizunara oak (Quercus crispula).

In some embodiments, the composition comprises Japanese oak (Quercus mongolica).

In some embodiments, the composition comprises Quercus pedunculata.

In some embodiments, the composition comprises Quercus sessiliflora.

In some embodiments, the composition comprises apple wood.

In some embodiments, the composition comprises cherry wood.

In some embodiments, the composition comprises maple wood.

In some embodiments, the composition comprises hickory wood.

In some embodiments, the composition comprises mesquite wood.

In some embodiments, the composition comprises pecan wood.

In some embodiments, the composition comprises alder wood.

In some embodiments, the composition comprises cypress wood.

In some embodiments, the composition comprises cedar.

In some embodiments, the composition comprises bourbon barrels, whiskey barrels, rum barrels, brandy barrels, wine barrels, madeira barrels, port barrels, tequila barrels, mescal barrels, sotol barrels, Cachaga barrels, or barrels that contained beer, wine, spirits, beverages or spices including, for example, sawdust obtained from any one or more of the foregoing.

In some embodiments, the composition comprises coniferous wood.

In some specific embodiments, the composition comprises Araucaria; hoop pine (Araucaria cunninghamii); monkey puzzle tree (Araucaria araucana); Parani pine (Araucaria angustifolia); cedar (Cedrus); celery-top pine (Phyllocladus aspleniifolius); cypress; Arizona cypress (Cupressus arizonica); bald cypress (Taxodium distichum); alerce (Fitzroya cupressoides); Hinoki cypress (Chamaecyparis obtusa); Lawson's cypress (Chamaecyparis lawsoniana); Mediterranean cypress (Cupressus sempervirens); Douglas fir (Pseudotsuga menziesii); European yew (Taxus baccata); fir (Abies); balsam fir (Abies balsamea); silver fir (Abies alba); noble fir (Abies procera); Pacific silver fir (Abies amabilis); hemlock (Tsuga); eastern hemlock (Tsuga canadensis); mountain hemlock (Tsuga mertensiana); western hemlock (Tsuga heterophylla); Huon pine (Lagarostrobos franklinii); kauri (Agathis australis); Queensland kauri (Agathis robusta); Japanese nutmeg-yew (Torreya nucifera); larch (Larix); European larch (Larix decidua); Japanese larch (Larix kaempferi); tamarack (Larix laricina); western larch (Larix occidentalis); pine (Pinus); European black pine (Pinus nigra); jack pine (Pinus banksiana); lodgepole pine (Pinus contorta); Monterey pine (Pinus radiata); Ponderosa pine (Pinus ponderosa); red pine (Pinus resinosa); Scots pine (Pinus sylvestris); white pine; eastern white pine (Pinus strobus); western white pine (Pinus monticola); sugar pine (Pinus lambertiana); southern yellow pine; loblolly pine (Pinus taeda); longleaf pine (Pinus palustris); pitch pine (Pinus rigida); shortleaf pine (Pinus echinata); red cedar; eastern red cedar (Juniperus virginiana); western red cedar (Thuja plicata); coast redwood (Sequoia sempervirens); rimu (Dacrydium cupressinum); spruce (Picea); Norway spruce (Picea abies); black spruce (Picea mariana); red spruce (Picea rubens); Sitka spruce (Picea sitchensis); white spruce (Picea glauca); sugi (Cryptomeria japonica); white cedar; northern white cedar (Thuja occidentalis); Atlantic white cedar (Chamaecyparis thyoides); African cypress (Widdringtonia species); pond cypress (Taxodium ascendens); Bald cypress (Taxodium distichum); Montezuma cypress (Taxodium mucronatum, Taxodium dubium); Chinese swamp cypress (Glyptostrobus pensilis); Cordilleran cypress (Austrocedrus chilensis); Cypress-pines; Cypress-pines (Callitris species); False cypress (Chamaecyparis species); Fujian cypress (Fokienia hodginsii); Guaitecas cypress (Pilgerodendron uviferum); Japanese cypress (Chamaecyparis obtusa); Patagonian cypress (Fitzroya cupressoides); Mediterranean cypress (Cupressus sempervirens); Monterey cypress (Cupressus macrocarpa); Nootka cypress (Cupressus nootkatensis); Siberian cypress (Microbiota decussata); Summer cypress (Bassia scoparia); Western red cedar (Thuja plicata); or nootka cypress (Cupressus nootkatensis).

In some embodiments, the composition comprises angiosperm wood.

In some specific embodiments, the composition comprises abachi (Triplochiton scleroxylon); acacia; African padauk (Pterocarpus soyauxii); afzelia (Afzelia africana); agba (Gossweilerodendron balsamiferum); alder (Alnus); black alder (Alnus glutinosa); red alder (Alnus rubra); ash (Fraxinus); black ash (Fraxinus nigra); blue ash (Fraxinus quadrangulata); common ash (Fraxinus excelsior); green ash (Fraxinus pennsylvanica); Oregon ash (Fraxinus latifolia); pumpkin ash (Fraxinus profunda); white ash (Fraxinus americana); aspen (Populus); bigtooth aspen (Populus gradidentata); European aspen (Populus tremula); quaking aspen (Populus tremuloides); Australian red cedar (Toona ciliata); ayan (Distemonanthus benthamianus); Agar wood; Aquilaria; balsa (Ochroma pyramidale); basswood; American basswood (Tilia americana); white basswood (Tilia heterophylla); American beech (Fagus grandifolia); birch (Betula); gray birch (Betula populifolia); black birch (Betula nigra); paper birch (Betula papyrifera); sweet birch (Betula lenta); yellow birch (Betula alleghaniensis); silver birch (Betula pendula); downy birch (Betula pubescens); blackbean (Castanospermum australe); blackwood; Australian blackwood (Acacia melanoxylon); African blackwood (Dalbergia melanoxylon); bloodwood (Brosimum rubescens); boxelder (Acer negundo); boxwood (Buxus sempervirens); Brazilian walnut (Ocotea porosa); brazilwood (Caesalpinia echinata); buckeye (Aesculus); horse-chestnut (Aesculus hippocastanum); Ohio buckeye (Aesculus glabra); yellow buckeye (Aesculus flava); butternut (Juglans cinerea); California bay laurel (Umbellularia californica); camphor tree (Cinnamomum camphora); cape chestnut (Calodendrum capense); catalpa (Catalpa); Ceylon satinwood (Chloroxylon swietenia); cherry (Prunus); black cherry (Prunus serotina); red cherry (Prunus pensylvanica); wild cherry (Prunus avium); chestnut (Castanea); chestnut (Castanea sativa); American chestnut (Castanea dentata); coachwood (Ceratopetalum apetalum); cocobolo (Dalbergia retusa); corkwood (Leitneria floridana); cottonwood; eastern cottonwood (Populus deltoides); swamp cottonwood (Populus heterophylla); cucumbertree (Magnolia acuminata); cumaru (Dipteryx); dogwood (Cornus); flowering dogwood (Cornus florida); Pacific dogwood (Cornus nuttallii); ebony (Diospyros); Andaman marblewood (Diospyros kurzii); ebene marbre (Diospyros melanida); African ebony (Diospyros crassiflora); Ceylon ebony (Diospyros ebenum); elm; American elm (Ulmus americana); English elm (Ulmus procera); rock elm (Ulmus thomasii); red elm (Ulmus rubra); wych elm (Ulmus glabra); eucalyptus; flooded gum (Eucalyptus grandis); white mahogany (Eucalyptus acmenoides); brown mallet (Eucalyptus astringens); southern mahogany (Eucalyptus botryoides); river red gum (Eucalyptus camaldulensis); karri (Eucalyptus diversicolor); blue gum (Eucalyptus globulus); rose gum (Eucalyptus grandis); york gum (Eucalyptus loxophleba); jarrah (Eucalyptus marginata); tallowwood (Eucalyptus microcorys); grey ironbark (Eucalyptus paniculata); blackbutt (Eucalyptus pilularis); mountain ash (Eucalyptus regnans); Australian oak (Eucalyptus obliqua); alpine ash (Eucalyptus delegatensis); red mahogany (Eucalyptus resinifera); swamp mahogany (Eucalyptus robusta); Sydney blue gum (Eucalyptus saligna); red ironbark (Eucalyptus sideroxylon); redwood (Eucalyptus transcontinentalis); Wandoo (Eucalyptus wandoo); European crabapple (Malus sylvestris); European pear (Pyrus communis); tigerwood (Astronium); greenheart (Chlorocardium rodiei); mpingo (Dalbergia melanoxylon); guanandi (Calophyllum brasiliense); gum (Eucalyptus); gumbo limbo (Bursera simaruba); hackberry (Celtis occidentalis); hickory (Carya); pecan (Carya illinoinensis); pignut hickory (Carya glabra); shagbark hickory (Carya ovata); shellbark hickory (Carya laciniosa); hornbeam (Carpinus); American hophornbeam (Ostrya virginiana); ipê (Handroanthus); African teak (Milicia excelsa); ironwood; balau (Shorea); American hornbeam (Carpinus caroliniana); sheoak (Casuarina equisetifolia); giant ironwood (Choricarpia subargentea); diesel tree (Copaifera langsdorffii); Borneo ironwood (Eusideroxylon zwageri); lignum vitae; guaiacwood (Guaiacum officinale); holywood (Guaiacum sanctum); takian (Hopea odorata); black ironwood (Krugiodendron ferreum); black ironwood (Olea); Lebombo ironwood (Androstachys johnsonii); Catalina ironwood (Lyonothamnus floribundus); Ceylon ironwood (Mesua ferrea); desert ironwood (Olneya tesota); Persian ironwood (Parrotia persica); Brazilian ironwood (Caesalpinia ferrea); yellow lapacho (Tabebuia serratifolia); jacarandi-boca-de-sapo (Jacaranda brasiliana); jacarandi de Brasil (Dalbergia nigra); jatobi (Hymenaea courbaril); kingwood (Dalbergia cearensis); lacewood; northern silky oak (Cardwellia sublimis); American sycamore (Platanus occidentalis); London plane (Platanus x acerifolia); limba (Terminalia superba); locust; black locust (Robinia pseudoacacia); honey locust (Gleditsia triacanthos); mahogany; genuine mahogany (Swietenia); West Indies mahogany (Swietenia mahagoni); bigleaf mahogany (Swietenia macrophylla); Pacific Coast mahogany (Swietenia humilis); African mahogany (Khaya); Chinese mahogany (Toona sinensis); Australian red cedar (Toona ciliata); Philippine mahogany (Toona calantas); Indonesian mahogany (Toona sureni); sapele (Entandrophragma cylindricum); sipo (Entandrophragma utile); tiama (Entandrophragma angolense); kosipo (Entandrophragma candollei); mountain mahogany (Entandrophragma caudatumi); Indian mahogany (Chukrasia velutina); Spanish Cedar (Cedrela odorata); light bosse (Guarea cedrata); dark bosse (Guarea thompsonii); American muskwood (Guarea grandifolia); carapa (Carapa guianensis); bead-tree (Melia azedarach); maple (Acer); hard maple; sugar maple (Acer saccharum); black maple (Acer nigrum); soft maple; boxelder (Acer negundo); red maple (Acer rubrum); silver maple (Acer saccharinum); European maple; sycamore maple (Acer pseudoplatanus); marblewood (Marmaroxylon racemosum); marri (Corymbia calophylla); meranti (Shorea); merbau (Intsia bijuga); mesquite; mopane (Colophospermum mopane); oak (Quercus); American oak or white oak (Quercus alba); bur oak (Quercus macrocarpa); post oak (Quercus stellata); swamp white oak (Quercus bicolor); southern live oak (Quercus virginiana); swamp chestnut oak (Quercus michauxii); chestnut oak (Quercus prinus); chinkapin oak (Quercus muhlenbergii); canyon live oak (Quercus chrysolepis); overcup oak (Quercus lyrata); red oak; northern red oak (Quercus rubra); eastern black oak (Quercus velutina); laurel oak (Quercus laurifolia); southern red oak (Quercus falcata); water oak (Quercus nigra); willow oak (Quercus phellos); Nuttall's oak (Quercus texana); okoume (Aucoumea klaineana); olive (Olea europaea); pink ivory (Berchemia zeyheri); poplar; balsam poplar (Populus balsamifera); black poplar (Populus nigra); hybrid black poplar (Populus x canadensis); purpleheart (Peltogyne); a species in the genus Prosopis; Queensland maple (Flindersia brayleyana); Queensland walnut (Endiandra palmerstonii); ramin (Gonystylus); redheart, chakte-coc (Erythroxylon mexicanum); sal (Shorea robusta); sweetgum (Liquidambar styraciflua); sandalwood (Santalum); Australian sandalwood (Santalum spicatum); Indian sandalwood (Santalum album); Hawaiian sandalwood (Santalum ellipticum, Santalum freycinetianum, Santalum paniculatum, Santalum haleakalae); Santalum acuminatum; Santalum yasi; Santalum spicatum; sassafras (Sassafras albidum); southern sassafras (Atherosperma moschatum); satine (Brosimum rubescens); silky oak (Grevillea robusta); silver wattle (Acacia dealbata); sourwood (Oxydendrum arboreum); Spanish-cedar (Cedrela odorata); Spanish elm (Cordia alliodora); tamboti (Spirostachys africana); teak (Tectona grandis); Thailand rosewood (Dalbergia cochinchinensis); tupelo (Nyssa); black tupelo (Nyssa sylvatica); tulip tree (Liriodendron tulipifera); turpentine (Syncarpia glomulifera); walnut (Juglans); Eastern black walnut (Juglans nigra); common walnut (Juglans regia); wenge (Millettia laurentii); panga-panga (Millettia stuhlmannii); willow (Salix); black willow (Salix nigra); cricket-bat willow (Salix alba Caerulea); white willow (Salix alba); weeping willow (Salix babylonica); or zingana (Microberlinia brazzavillensis).

In some specific embodiments, the composition comprises Amburana; Amburana acreana; Amburana cearensis; Amburana erythrosperma; Apple; Malus domestica; Malus sieversii; Palo Santo (Bursera graveolens); clove (Syzygium aromaticum); star anise (Illicium verum); cinnamon; Ceylon cinnamon (Cinnamomum verum); Cinnamomum burmannii; Cinnamomum cassia; Cinnamomum loureiroi; Cinnamomum citriodorum; Brazilian rosewood (Dalbergia nigra); cocobolo (Dalbergia retusa); lignum vitae (Guaiacum officinale); raspberry jam wood (Acacia acuminata); Torreya; Torreya californica; Torreya fargesii; Torreya grandis; Torreya jackii; Torreya nucifera; Torreya taxifolia; thuya (Tetraclinis articulata); cacao wood (Theobroma cacao); peach (Prunus persica); apricot (Prunus armeniaca; Prunus brigantina; Prunus cathayana; Prunus dasycarpa; Prunus hongpingensis; Prunus hypotrichodes; Prunus limeixing; Prunus mandshurica; Prunus mume; Prunus sibirica; Prunus zhengheensis); plum (Prunus domestica; Prunus salicina; Prunus simonii); almond; Prunus amygdalus; Prunus dulcis; pistachio (Pistacia vera); honey mesquite (Prosopis glandulosa); velvet mesquite (Prosopis velutina); mesquite (Prosopis spp, Prosopis pallida or Prosopis juliflora); creosote bush (Larrea tridentata); mulberry (Morus); crabapple (Malus sylvestris); or a species of magnolia (Magnoliaceae); In some embodiments, the composition comprises biomass of a mute plant.

In some specific embodiments, the mute plant is lily of the valley (Convallaria), lilac (Syringa), honeysuckle (Lonicera), violet (Violaceae), seringa (Philadephaceae), hyacinth (Hyacinthus), sweet pea (Lathyrus), or a species of Magnolia (Magnoliaceae) flower. In some very specific embodiments, the composition comprises biomass of lily of the valley.

In some embodiments, the composition comprises plum blossoms, cherry blossoms, apple blossoms, orange blossoms, lemon blossoms, lime blossoms, satsuma blossoms, osmanthus blossoms, jasmine blossoms, Frangipani (Plumeria) blossoms, Nyctanthes arbor-tristis, lavender, tuberose flowers, lilies, rose, rose blossoms, ylang ylang (Cananga odorata), Manoranjitham (Artabotrys hexapetalus), Narcissus flowers, Scented Primrose (Primula vulgaris), Sweet Autumn Clematis (Clematis terniflora), Nicotiana (Nicotiana), Viburnum, Mock Orange (Philadelphus), Lilac (Syringa), Angel's Trumpet (Brugmansia), Daphne, Night Scented Stocks (Matthiola longipetala), Magnolia, Brunfelsia pauciflora, Freesia, Wrightia religiosa, Hedychium coronarium, Fagraea berteroana, Tabernaemontana divaricate, a species of magnolia (Magnoliaceae), Magnolia champaca, Cestrum nocturnum, Gardenia, Wisteria, Azalia, Sweet osmanthus, Camellia, Sasanqua camellia, or Magnolia grandiflora.

In some embodiments, the composition comprises Agarwood: oud; Aquilaria; Aquilaria malaccensis; frankincense; Boswellia; Boswellia sacra; Boswellia bhaw-dajiana; Boswellia carteri; Boswellia frereana, Boswellia serrata; Boswellia thurifera; Boswellia papyrifera; Galbanum; Ferula; Ferula gummosa; Ferula rubricaulis; orris; Rhizoma iridis; Iris germanica; Iris pallida; a species in the genus Iridaceae; lis root, iris root; Amber; Baltic Amber; Ambergris; Ambrette; Ambrette seeds; Amyris; Balsamic; benzoin; pine; resin; juniper; turpentine; Styrax tree; Bergamot; bergamot orange; Clone; Cashmeran; galbanum resin; Guaiac Wood; Hedione; Heliotrope; flowers from the heliotropism family; vetiver; vetiver roots; patchouli; patchouli leaves; patchouli bush; Indole, Iso-E-Super, Jasmine; Coconut; Labdanum; rockrose bush; Leather; Myrrh; Commiphora; Narcissus; Oakmoss; lichen; Opopanax; Sweet Myrrh; balsam; Osmanthus; Rose; Clary sage; or Tonka bean.

In some embodiments, the composition comprises allspice (Pimenta dioica); angelica (Angelica archangelica); anise (Pimpinella anisum); asafoetida (Ferula assa-foetida); bay leaf (Laurus nobilis); basil (Ocimum basilicum); bergamot (Monarda species); black cumin (Nigella sativa); black mustard (Brassica nigra); black pepper (Piper nigrum); borage (Borago officinalis); brown mustard (Brassica juncea); burnet (Sanguisorba minor and S. officinalis); caraway (Carum carvi); cardamom (Elettaria cardamomum); cassia (Cinnamomum cassia); catnip (Nepeta cataria); cayenne pepper (Capsicum annuum); celery seed (Apium graveolens, variety dulce); chervil (Anthriscus cerefolium); chicory (Cichorium intybus); chili pepper (Capsicum species); chives (Allium schoenoprasum); cicely (Myrrhis odorata); cilantro (Coriandrum sativum); cinnamon (Cinnamomum verum); clove (Syzygium aromaticum); coriander (Coriandrum sativum); costmary (Tanacetum balsamita); cumin (Cuminum cyminum); curry; dill (Anethum graveolens); fennel (Foeniculum vulgare); fenugreek (Trigonella foenum-graecum); file (Sassafras albidum); ginger (Zingiber officinale); grains of paradise (Aframomum melegueta); holy basil (Ocimum tenuiflorum); horehound (Marrubium vulgare); horseradish (Armoracia rusticana); hyssop (Hyssopus officinalis); lavender (Lavandula species); lemon balm (Melissa officinalis); lemon grass (Cymbopogon citratus); lemon verbena (Aloysia citrodora); licorice (Glycyrrhiza glabra); lovage (Levisticum officinale); mace (Myristica fragrans); marjoram (Origanum majorana); nutmeg (Myristica fragrans); oregano (Origanum vulgare); paprika (Capsicum annuum); parsley (Petroselinum crispum); peppermint (Mentha xpiperita); poppy seed (Papaver somniferum); rosemary (Salvia rosmarinus); rue (Ruta graveolens); saffron (Crocus sativus); sage (Salvia officinalis); savory (Satureja hortensis and S. montana); sesame (Sesamum indicum); sorrel (Rumex species); star anise (Illicium verum); spearmint (Mentha spicata); tarragon (Artemisia dracunculus); thyme (Thymus vulgaris); turmeric (Curcuma longa); vanilla (Vanilla planifolia and V. tahitensis); wasabi (Eutrema japonicum); or white mustard (Sinapis alba).

In some embodiments, the composition comprises saffron.

In some embodiments, the composition comprises vanilla.

In some embodiments, the composition comprises cinnamon.

In some embodiments, the composition comprises capsaicin.

In some embodiments, the composition comprises menthol.

In some embodiments, the composition comprises a type of peppercorn.

In some embodiments, the composition comprises leather.

In some embodiments, the composition comprises a plant species from the genus Nicotiana.

In some embodiments, the composition comprises Nicotiana acuminata; Nicotiana Africana; Nicotiana alata; Nicotiana attenuata; Nicotiana benthamiana; Nicotiana clevelandii; Nicotiana glauca; Nicotiana glutinosa; Nicotiana langsdorffii; Nicotiana longiflora; Nicotiana occidentalis; Nicotiana obtusifolia; Nicotiana otophora; Nicotiana plumbaginifolia; Nicotiana quadrivalvis; Nicotiana rustica; Nicotiana suaveolens; Nicotiana sylvestris; Nicotiana tabacum; Nicotiana tomentosiformis; Nicotiana x didepta; Nicotiana debneyi x Nicotiana. tabacum; Nicotiana x digluta; Nicotiana glutinosa x Nicotiana tabacum; Nicotiana x sanderae; or Nicotiana alata x Nicotiana forgetiana.

In some embodiments, the composition comprises tea.

In some embodiments, the composition comprises one or more of the following types of tea: Green tea; Chun Mee; Chun Lu; Bi Luo Chun; Gunpowder; Maofeng; Yellow; Jasmine; Anji Bai Cha; Maojian; Taiping Houkui; Jin Shan; Longjing (Dragon Well); Sejak; Ujeon; Jungjak; Daejak; Sencha; Gyokuro; Kabusecha; Tencha; Matcha; Mecha; Shincha; Hojicha; Kukicha; Bancha; Genmaicha; Konacha; Kamairicha; Tamaryokucha; Black tea; Assam; English Breakfast; Earl Grey; Darjeeling; Rukeri; Pu-Erh; Scottish Afternoon; Irish Breakfast; Milima; Ceylon; Chai; Panyang Congou; Keemun; Lapsang Souchong; Golden Tips; Temi Sikkim; Nimbu; Wakuocha; White tea; Silver Needle; White Peony; Shou Mei; Gong Mei; Darjeeling White; Oolong tea; Da Hong Pao; Shui Jin Gui; Tie Luo Han; Shui Xian; Bai Jiguan; Tieguanyi (Iron Goddess); Mi Lan Xiang Dan Con; Ancient Tree Dan Cong; Guan Yin; Dancong; Cassia; Da Yu Lin; Dong Ding; Dong Fang Meiren; Alishan; Pouchong; Ruan Zhi; Jin Xuan; or Li Shan.

In some embodiments, the composition comprises a herbal tea.

In some embodiments, the composition comprises one or more of the following types of tea Avocado Leaf, Bamboo; Butterfly Pea Flower; Chaga Mushroom; Chamomile; Lavender; Liquorish; Guayusa; Honeysuckle Flower; Lemon; Mint; Olive Leaves; Hibiscus; Rooibos; Turmeric; Pumpkin Spice; Chrysanthemum; Buckwheat; Honeybush; Bush; Mamaki; Yaupon; or Yerba mate.

In some embodiments, the composition comprises yerba mate.

In some embodiments, the composition comprises rooibos.

In some embodiments, the composition comprises meat or dried meat.

In some embodiments, the composition comprises dried mushrooms.

In some embodiments, the composition comprises beans.

In some embodiments, the composition comprises soybeans.

In some embodiments, the composition comprises fermented plants.

In some embodiments, the composition comprises dried fermented plants.

In some embodiments, condensing the vaporized molecule into a condensed molecule comprises contacting the vaporized molecule with a solvent. In some specific embodiments, condensing the vaporized molecule into a condensed molecule comprises contacting the vaporized molecule with a collection solvent.

In some embodiments, the condensed molecule is dissolved in a solvent.

In some embodiments, the solvent is ethanol.

In some embodiments, the solvent is water.

In some embodiments, the solvent is propylene glycol.

In some embodiments, the solvent is glycerol.

In some embodiments, the solvent is a triglyceride.

In some embodiments, condensing the vaporized molecule into the condensed molecule comprises condensing a plurality of vaporized molecules that comprises the vaporized molecule into a distillate that comprises the condensed molecule.

Various aspects of this disclosure relate to a distillate produced according to a method described anywhere in the disclosure, wherein the distillate is an essential oil of the composition, and the distillate comprises the condensed molecule.

Various aspects of this disclosure relate to a product manufactured from a distillate described in either of the two preceding paragraphs.

In some embodiments, the product is a beverage.

In some specific embodiments, the product is an alcoholic beverage. In some very specific embodiments, the product is a liquor, wine, beer, or cocktail.

In some embodiments, the product is a non-alcoholic cocktail.

In some embodiments, the product is a bottled, ready-to-drink cocktail.

In some embodiments, the product is a wine. In some specific embodiments, the product is a chardonnay; Cabernet Sauvignon; Syrah; Zinfandel; Pinot Noir; Sauvignon Blanc; Pinot Gris; Riesling; Merlot; Rose; Port; or Madeira.

In some embodiments, the product is a liquor.

In some specific embodiments, the product is a whiskey, a bourbon, a scotch, an Irish whiskey, a blended whiskey, a rye whiskey, a corn whiskey, or a Canadian whiskey.

In some specific embodiments, the product is a rum, a brandy, a gin, a tequila, a mescal, a sotol, a vodka, or another type of distilled spirit.

In some embodiments, the product is a consumer packaged good.

In some embodiments, the product is a flavoring.

In some embodiments, the product is a mixology product.

In some embodiments, the product is an aromatic cocktail garnish.

In some embodiments, the product is a cocktail spray.

In some embodiments, the product is a bitters.

In some embodiments, the product is an edible extract that is classified as generally regarded as safe (GRAS) by the United States Food and Drug Administration (FDA).

In some embodiments, the product is a flavor that is classified as GRAS by the FDA.

In some embodiments, the product is a United States Alcohol and Tobacco Tax and Trade Bureau approved flavor.

In some embodiments, the product is a United States Alcohol and Tobacco Tax and Trade Bureau approved extract.

In some specific embodiments, the product is a nonalcoholic liquor, wine, beer, or cocktail that contains less than 0.5% alcohol by volume.

In some embodiments, the product is a tannin solution.

In some embodiments, the product is synthetic vanillin.

In some embodiments, the product is a food sauce.

In some embodiments, the product is a food.

In some embodiments, the product is a dietary supplement.

In some embodiments, the product is a fragrance.

In some embodiments, the product is a scented skin care product.

In some embodiments, the product is a perfume.

In some embodiments, the product is an air freshener.

In some embodiments, the product is a cleaning preparation.

In some embodiments, the product is a soap or detergent.

In some embodiments, the product is a scented candle.

In some embodiments, the product is an incense.

In some embodiments, the product is a scented candle.

In some embodiments, the product is a tobacco flavoring.

In some embodiments, the product is hookah flavoring.

In some embodiments, the product is an essential oil.

In some embodiments, the product is a medicine.

The following examples provide a framework to implement certain aspects of the disclosure, and these examples do not limit the scope of this patent document or any claim that matures from the disclosure of this patent document.

### Example 1: Extraction of Coumarin from Amburana Wood Barrels

The wood of Amburana cearensis is known to contain coumarin. Amburana wood barrels are commonly used to age Cachaga, a distilled spirit that has similar qualities to rum. Coumarin is the primary source of aroma in Amburana wood, but it is only present in small quantities, making it exceptionally difficult to extract using conventional solvent methods.

Three kilograms of medium-toast Amburana cearensis wood was ground and extracted using an extraction machine as described in European Patent No. 3,283,606 B1. The extraction machine was operated with an extraction temperature that did not exceed 205 degrees Celsius. The vaporized coumarin was collected in three liters of ethanol. 100 milliliters of the resulting extract was analyzed using high-pressure liquid chromatography (HPLC). The extract was found to contain 1,138 milligrams of coumarin per kilogram.

Based on known quantities of coumarin in toasted Amburana cearensis, the method of the present disclosure efficiently extracted coumarin at an extraction temperature far below its known boiling point. Coumarin has a boiling point of 301.7 degrees Celsius, yet the extraction machine never exceeded an extraction temperature of 205 degrees Celsius.

### Example 2: Extraction of Toasted French Oak

Toasted French oak is known to contain a just few milligrams per kilogram of vanillin, eugenol, and isoeugenol, yet these compounds make a profound contribution to barrel-aged whiskey and other barrel-aged spirits.

Three kilograms of medium-toast French oak was ground and extracted using an extraction machine as described in European Patent No. 3,283,606 B1. The extraction machine was operated at an extraction temperature that did not exceed 205 degrees Celsius. The vaporized vanillin, eugenol, and isoeugenol was collected in three kilograms of a collection solvent consisting of ethanol and water. 100 milliliters of the resulting extract was analyzed using a gas chromatography mass spectrometer (GC-MS) and found to contain 15.8 milligrams of vanillin per kilogram, 0.2 milligrams of eugenol per kilogram, and 0.9 milligrams of isoeugenol per kilogram.

Based on the known quantities of vanillin, eugenol, and isoeugenol in toasted French oak, the method of the present disclosure efficiently extracted each of these compounds at temperatures far below their known boiling points. Vanillin has a boiling point of 285 degrees Celsius, yet the extraction machine never exceeded an extraction temperature of 205 degrees Celsius. Eugenol has a boiling point of 252 degrees Celsius, yet the extraction machine never exceeded an extraction temperature of 205 degrees Celsius. Isoeugenol has a boiling point of 266 degrees Celsius, yet the extraction machine never exceeded an extraction temperature of 205 degrees Celsius.

### Example 3: Extraction of Toasted American Oak

Toasted American oak is known to contain a just few milligrams per kilogram of vanillin, eugenol, and isoeugenol, yet these compounds make a profound contribution to barrel-aged whiskey and other barrel-aged spirits.

Three kilograms of dark-toast American oak was ground and extracted using an extraction machine as described in European Patent No. 3,283,606 B1. The extraction machine was operated with an extraction temperature that did not exceed 205 degrees Celsius. The vaporized vanillin, eugenol, and isoeugenol was collected in three kilograms of a collection solvent consisting of ethanol and water. The resulting extract was analyzed using GC-MS methods and was found to contain 13.1 milligrams of vanillin per kilogram, 0.9 milligrams of eugenol per kilogram, and 2.4 milligrams of isoeugenol per kilogram.

Based on the known quantities of these compounds in toasted American oak, the method of the present disclosure efficiently extracted each of these compounds at temperatures far below their known boiling points. Vanillin has a boiling point of 285 degrees Celsius, yet the extraction machine never exceeded an extraction temperature of 205 degrees Celsius. Eugenol has a boiling point of 252 degrees Celsius, yet the extraction machine never exceeded an extraction temperature of 205 degrees Celsius. Isoeugenol has a boiling point of 266 degrees Celsius, yet the extraction machine never exceeded an extraction temperature of 205 degrees Celsius.

### Example 4: Extraction of Sandalwood

Sandalwood derives its signature scent from alpha-santalol, which boils at 302 degrees Celsius.

Three kilograms of sandalwood is finely ground to increase its surface area. The ground sandalwood is homogenized by mixing to ensure consistency throughout. A randomized 100-gram sample of the homogenized sandalwood is tested using HPLC to analyze its starting alpha-santalol content. The test results indicate that the pre-extraction sandalwood contains approximately 33,000 milligrams of alpha-santalol per kilogram of sandalwood.

The remaining 2,900 grams of sandalwood is extracted using the method of the present disclosure at a sweep gas temperature of 210 degrees Celsius and a pressure of 760.00 mm Hg. The vaporized alpha-santalol is captured in three kilograms of a collection solvent consisting of ethanol and water to produce a highly aromatic sandalwood extract.

100 milliliters of the extract is homogenized and tested using HPLC methods. The test results indicate that the extract contains 23,430 milligrams per kilogram. The depleted post-extraction sandalwood is homogenized by mixing, and a 100-gram sample is tested using HPLC methods. The test results indicate that the post-extraction sandalwood contains only 7,260 milligrams of alpha-santalol per kilogram of sandalwood.

Alpha-santalol has a boiling point of 302 degrees Celsius, yet efficient extraction is achieved at 210 degrees Celsius. A comparison of the HPLC results for the pre-extraction sandalwood and the post-extraction sandalwood indicates that approximately 78% of the available alpha-santalol has been removed from the sandalwood. A comparison of the HPLC results for the pre-extraction sandalwood and the extract indicates that approximately 76% of the available alpha-santalol is captured from the sandalwood. Approximately 2% of the total alpha-santalol is unaccounted for.

### Example 5: Extraction of Sandalwood

Three kilograms of ground sandalwood were ground and extracted using the methods of the present invention with a sweep gas temperature of 200 degrees Celsius. A highly aromatic sandalwood extract was produced that was judged by several senior fragrance industry experts to have greater aromatic intensity than steam-distilled and solvent-extracted reference samples. A sensory panel was conducted on the sandalwood source material before and after extraction using the method of the present disclosure. The starting source material displayed a strong aroma of freshly ground sandalwood. The extracted source material possessed little remaining aroma. This experiment was repeated at a sweep gas temperature of 170 Celsius, 180 Celsius, and 210 Celsius. In all cases, most of the aroma associated with alpha-santalol was stripped from the starting source material.

### Example 6: Extraction of Orris

Orris root derives its signature scent from irone, which boils at 295 degrees Celsius.

Three kilograms of orris is finely ground to increase its surface area. The ground orris is homogenized by mixing to ensure consistency throughout. A randomized 100-gram sample of the homogenized orris is tested using HPLC methods to analyze its starting irone content. The test results indicate that the pre-extraction orris contains approximately 1,880 milligrams of irone per kilogram of orris.

The remaining 2,900 grams of orris is extracted using the method of the present disclosure at a sweep gas temperature of 200 degrees Celsius and a pressure of 760.00 mm Hg. The vaporized irone is captured in three kilograms of a collection solvent consisting of ethanol and water to produce a highly aromatic orris extract.

100 milliliters of the extract is tested using HPLC methods. The test results indicate that the extract contains 1,523 milligrams of irone per kilogram. The depleted post-extraction orris is homogenized by mixing, and a 100-gram sample is tested using HPLC methods. The test results indicate that the post-extraction orris contains only 301 milligrams of irone per kilogram of orris.

Irone has a boiling point of 295 degrees Celsius, yet efficient extraction is achieved at 200 degrees Celsius. A comparison of the HPLC results for the pre-extraction orris and the post-extraction orris indicates that approximately 84% of the available irone has been removed from the orris. A comparison of the HPLC results for the pre-extraction orris and the extract indicates that approximately 81% of the available irone is captured from the orris. Approximately 3% of the total irone is unaccounted for.

### Example 7: Extraction of Orris

Three experiments were performed using ground orris as a source material. For each experiment, three kilograms of ground orris root was extracted using the methods of the present invention. In the first experiment, a sweep gas temperature of 170 degrees Celsius was utilized. In the second experiment a sweep gas temperature of 190 degrees Celsius was utilized. In the third experiment, a sweep gas temperature of 205 degrees Celsius was utilized. In each case, a highly aromatic orris extract was produced. Several senior fragrance industry experts determined that the orris extract produced with the methods of the present disclosure were preferred over steam-distilled and solvent-extracted reference samples. For each experiment, a sensory panel was conducted on the source material before and after extraction using the method of the present disclosure. The starting source material displayed a strong aroma of freshly-ground orris. For all experiments, the extracted source material was found to possess little remaining aroma, with only slightly more aroma present in the 170 degrees Celsius extracted source material compared to the 205 degrees Celsius extracted source material. In each case, the vast majority of the irone present in the orris had apparently been removed from the source material and deposited into the extract.

### Example 8: Extraction of Various Woods and Detection of High-Boiling Point Compounds

Several different compositions consisting of different types of wood, wood barrels that formerly contained distilled spirits, and wood barrels that formerly contained wine, were each separately ground into sawdust particles measuring less than 1 millimeter in length on average. In a series of separate tests, the wood particles were extracted using methods of this disclosure, and GC-MS analyses were performed on the resulting extracts. The experiments were performed in an extraction machine as described in European Patent No. 3,283,606 B1. For each test, the sweep gas was heated to approximately 205 degrees Celsius. The wood particles were introduced continuously to an extraction chamber of the extraction machine at a metered rate by an auger. Upon entering the extraction chamber, the wood particles were bombarded with the sweep gas. The bombardment of the sweep gas aerosolized the wood particles and transported the wood particles through the extraction chamber along with the sweep gas. The aerosolized composition remained in contact with the sweep gas for several seconds as it passed through the length of the extraction chamber. The extraction chamber included turns, to create turbulent air flow to increase the mass transfer rate of molecules of the composition into vaporized molecules. In each test, molecules were evaporated from each of the different compositions to form a vapor. A cyclone separator was used to separate the vaporized molecules evaporated vapor from the non-evaporated components of the wood particles. The sweep gas containing the separated vaporized molecules was passed through a spray of collection solvent to condense the molecules into condensed molecules. The collection solvent contained a blend of ethanol and water to attract, absorb and hold the molecules. To reach the desired concentration of condensed molecules in the collection solvent, in each test, the spray of collection solvent was continuously recirculated by a liquid pump, and the sweep gas containing vaporized molecules was passed continuously passed through the spray of collection solvent. The embodiment was operated continuously until approximately three kilograms of wood particles had passed through the extraction machine. In each case, an aromatic wood extract was captured in the collection solvent. 100 milliliters of each extract was analyzed using GC-MS and HPLC methods. Even though the sweep gas and extraction chamber were held at or below approximately 205 degrees Celsius, significant quantities of the following higher-boiling-point molecules were detected in the different tests: furyl-hydroxymethyl ketone (CAS: 17678-19-2), which boils a 239 degrees Celsius; 2,5-furandicarboxaldehyde (CAS: 823-82-5) which boils at 276 to 277 degrees Celsius; nonan-1-ol (CAS: 143-08-8), which boils at 214 degrees Celsius; decan-1-ol (CAS: 112-53-8), which boils at 231 degrees Celsius; dodec-1-ol (CAS: 112-53-8), which boils at 230 degrees Celsius; tetradecane-1-ol (CAS: 112-72-1), which boils at 289 degrees Celsius; hexadecane-1-ol (CAS: 36653-82-4), which boils at 344 degrees Celsius; octadecane-1-ol (CAS: 112-92-5) which boils at 384 degrees Celsius; 2-decen-1-ol (CAS: 22104-80-9) which boils at 229 degrees Celsius; phenylethyl alcohol (CAS: 60-12-8), which boils at 219-221 degrees Celsius; tetradecanal (CAS: 124-25-4), which boils at 260 degrees Celsius; (Z)-2-decenal (CAS: 2497-25-8), which boils at 226-230 degrees Celsius; (E,E)-2,4-decadienal (CAS: 25152-84-5), which boils at 279 to 280 degrees Celsius; octanoic acid (CAS: 124-07-2), which boils at 237 degrees Celsius; nonanoic acid (CAS: 112-05-0), which boils at 254 degrees Celsius; n-decanoic acid (CAS: 334-48-5), which boils at 268.00 to 270.00 degrees Celsius; n-hexadecanoic acid (CAS: 57-10-3), which boils at 351 degrees Celsius; heptadecanoic acid (CAS: 506-12-7), which boils at 263 degrees Celsius; octadecanoic acid (CAS: 57-11-4), which boils at 361 degrees Celsius; 2-ethylhexanoic acid (CAS: 149-57-5), which boils at 228 degrees Celsius; trans-2-undecenoic acid (CAS: 15790-94-0), which boils at 295 degrees Celsius; benzoic acid (CAS: 65-85-0), which boils at 249 degrees Celsius; phthalic acid (CAS: 88-99-3), which boils at 289 degrees Celsius; methyl nonanoate (CAS: 1731-84-6), which boils at 213 degrees Celsius; octyl butanoate (CAS: 110-39-4), which boils at 224 degrees Celsius; isopropyl myristate (CAS: 110-27-0), which boils at 315 degrees Celsius; ethyl nicotinate (CAS: 614-18-6), which boils at 224 degrees Celsius; 3-hexenyl butanoate (CAS: 53398-84-8), which boils at 213 degrees Celsius; diethyl butanedioate (CAS: 123-25-1), which boils at 217 degrees Celsius; diethyl itaconate (CAS: 2409-52-1), which boils at 213 degrees Celsius; benzyl benzoate (CAS 120-51-4), which boils at 323 degrees Celsius; methyl salicylate (CAS: 119-36-8), which boils at 222-224 degrees Celsius; eugenol (CAS 97-53-0), which boils at 252-253 degrees Celsius; vanillin (CAS 121-33-5), which boils at 285-286 degrees Celsius; (Z)-isoeugenol (CAS: 5932-68-3), which boils at 266-268 degrees Celsius; ethyl anisate (CAS: 94-30-4), which boils at 263 degrees Celsius; 4-methylguauacol (CAS: 93-51-6), which boils at 221 degrees Celsius; (E)-2,6-dimethoxy-4-(prop-1-en-1-yl)phenol (CAS: 20675-95-0), which boils at 305 degrees Celsius; thymol (CAS: 89-83-8), which boils at 232 degrees Celsius; caryophyllene (CAS: 87-44-5), which boils at 256-259 degrees Celsius; α-bisabolol (CAS 515-69-5), which boils at 314-315 degrees Celsius; (4R,5R)-5-butyl-4-methyloxolan-2-one (CAS: 55013-32-6), which boils at 245-247 degrees Celsius; (4S,5R)-5-butyl-4-methyloxolan-2-one (CAS: 39638-67-0), which boils at 246 degrees Celsius; β-ionone (CAS: 8013-90-9), which boils at 255 degrees Celsius; 6-tetradecalactone (CAS: 2721-22-4), which boils at 322 degrees Celsius; bis(2-furfuryl)disulfide (CAS: 4437-20-1), which boils at 229-230 degrees Celsius; hydrocoumarin (CAS: 119-84-6), which boils at 272 degrees Celsius; and coumarin (CAS: 91-64-5), which boils at 301.7 degrees Celsius. In most cases, the mass extracted of the above molecules represented corresponded to a majority of the mass known to present in the starting wood compositions. Several hundred other compounds were also found in testing that have not been mentioned.

What is claimed is:

1. A method to separate a molecule from an impurity, comprising:
providing a system to separate the molecule from the impurity;
providing a composition comprising the molecule and the impurity, wherein the molecule is vanillin, the vanillin is present in the composition in a solid phase or a liquid phase, the impurity is cellulose I, and the impurity is present in the composition in a solid phase or a liquid phase;
converting the molecule into a vaporized molecule in a gas phase, wherein converting the molecule into the vaporized molecule is performed in a first chamber of the system, the gas phase has a pressure and a temperature, the molecule has a boiling point at the pressure and a vapor pressure at the temperature, the pressure of the gas phase is greater than the vapor pressure of the molecule, the boiling point of the molecule is greater than the temperature of the gas phase, and either the impurity lacks a vapor pressure or the impurity has a vapor pressure at the temperature that is less than the vapor pressure of the molecule at the temperature;
separating the vaporized molecule from the impurity; and
condensing the vaporized molecule into a condensed molecule in a second chamber of the system,
wherein:
the system allows passage of the gas phase from the first chamber to the second chamber;
the system inhibits passage of the impurity from the first chamber to the second chamber;
the composition comprises biological cells;
the biological cells have interiors, exteriors, and cell membranes that separate the interiors from the exteriors;
the method comprises vaporizing an accessory molecule within the biological cells at a rate sufficient to generate pressure within the biological cells that ruptures at least 10 percent of the cell membranes;
the composition off-gasses the vaporized molecule at a vaporization rate;
the rupturing increases the vaporization rate;
the vaporized molecule recondenses onto the composition at a condensation rate;
converting the molecule into the vaporized molecule occurs at a mass transfer rate, which is equal to the vaporization rate minus the condensation rate;
the rupturing increases the mass transfer rate;
the accessory molecule is water; and
the temperature of the gas phase is greater than the boiling point of the water at the pressure of the gas phase.

2. A method to separate a molecule from an impurity, comprising:
providing a system to separate the molecule from the impurity;
providing a composition comprising the molecule and the impurity, wherein the molecule is vanillin, the vanillin is present in the composition in a solid phase or a liquid phase, the impurity is cellulose I, and the impurity is present in the composition in a solid phase or a liquid phase;
converting the molecule into a vaporized molecule in a gas phase, wherein converting the molecule into the vaporized molecule is performed in a first chamber of the system, the gas phase has a pressure and a temperature, the molecule has a boiling point at the pressure and a vapor pressure at the temperature, the pressure of the gas phase is greater than the vapor pressure of the molecule, the boiling point of the molecule is greater than the temperature of the gas phase, and either the impurity lacks a vapor pressure or the impurity has a vapor pressure at the temperature that is less than the vapor pressure of the molecule at the temperature;
separating the vaporized molecule from the impurity; and
condensing the vaporized molecule into a condensed molecule in a second chamber of the system,
wherein:
the system allows passage of the gas phase from the first chamber to the second chamber;
the system inhibits passage of the impurity from the first chamber to the second chamber;
the composition comprises biological cells;
the biological cells have interiors, exteriors, and cell membranes that separate the interiors from the exteriors; and
the method comprises vaporizing an accessory molecule within the biological cells at a rate sufficient to generate pressure within the biological cells that ruptures a portion of the cell membranes.

3. A method to separate a molecule from an impurity, comprising:
providing a system to separate the molecule from the impurity;
providing a composition comprising the molecule and the impurity, wherein the molecule is vanillin, the vanillin is present in the composition in a solid phase or a liquid phase, the impurity is cellulose I, and the impurity is present in the composition in a solid phase or a liquid phase;
converting the molecule into a vaporized molecule in a gas phase, wherein converting the molecule into the vaporized molecule is performed in a first chamber of the system, the gas phase has a pressure and a temperature, the molecule has a boiling point at the pressure and a vapor pressure at the temperature, the pressure of the gas phase is greater than the vapor pressure of the molecule, the boiling point of the molecule is greater than the temperature of the gas phase, and either the impurity lacks a vapor pressure or the impurity has a vapor pressure at the temperature that is less than the vapor pressure of the molecule at the temperature;
separating the vaporized molecule from the impurity; and
condensing the vaporized molecule into a condensed molecule in a second chamber of the system,
wherein:
the system allows passage of the gas phase from the first chamber to the second chamber;
the system inhibits passage of the impurity from the first chamber to the second chamber;
the composition comprises biological cells;
the biological cells have interiors, exteriors, and cell membranes that separate the interiors from the exteriors;
the method comprises vaporizing an accessory molecule within the biological cells at a rate sufficient to generate pressure within the biological cells that ruptures at least 10 percent of the cell membranes;
the accessory molecule is water; and
the temperature of the gas phase is greater than the boiling point of the water at the pressure of the gas phase.

4. The method of claim 3, comprising:
feeding the composition into the first chamber of the system at a feed rate, which is the amount of the molecule that is fed into the first chamber per unit time;
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the latent heat transfer has a rate;
the bombarding increases the rate of the latent heat transfer;
the composition has a surface-area-to-volume ratio that is greater than 500 per meter;
the surface-area-to-volume ratio of the composition results in a mass transfer rate of at least 5 micrograms of the molecule per gram of the composition per second at the temperature and the pressure of the gas phase;
the composition off-gasses the vaporized molecule at a vaporization rate;
a greater surface-area-to-volume ratio correlates with a greater vaporization rate;
the method comprises condensing the vaporized molecule into the condensed molecule from a first portion of the composition in the second chamber and concurrently converting the molecule into the vaporized molecule from a subsequent portion of the composition in the first chamber;
the method is performed such that the collection rate is at least 50 percent and no greater than 100 percent of the mass transfer rate over a period of time;
the mass transfer rate is at least 50 percent and no greater than 100 percent of the feed rate over a concurrent period of time; and
the period of time and the concurrent period of time are the same 10 second period.

5. The method of claim 3, comprising:
feeding the composition into the first chamber of the system at a feed rate, which is the amount of the molecule that is fed into the first chamber per unit time;
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the latent heat transfer has a rate;
the bombarding increases the rate of the latent heat transfer;
the composition has a surface-area-to-volume ratio that is greater than 500 per meter;
the composition off-gasses the vaporized molecule at a vaporization rate;
a greater surface-area-to-volume ratio correlates with a greater vaporization rate;
the method comprises condensing the vaporized molecule into the condensed molecule from a first portion of the composition in the second chamber and concurrently converting the molecule into the vaporized molecule from a subsequent portion of the composition in the first chamber;
the method is performed such that the collection rate is at least 50 percent and no greater than 100 percent of the mass transfer rate over a period of time;
the mass transfer rate is at least 50 percent and no greater than 100 percent of the feed rate over a concurrent period of time; and
the period of time and the concurrent period of time are the same 10 second period.

6. The method of claim 3, comprising:
feeding the composition into the first chamber of the system at a feed rate, which is the amount of the molecule that is fed into the first chamber per unit time;
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the latent heat transfer has a rate;
the bombarding increases the rate of the latent heat transfer;
the composition has a surface-area-to-volume ratio that is greater than 500 per meter;
the composition off-gasses the vaporized molecule at a vaporization rate;
the method comprises condensing the vaporized molecule into the condensed molecule from a first portion of the composition in the second chamber and concurrently converting the molecule into the vaporized molecule from a subsequent portion of the composition in the first chamber;
the method is performed such that the collection rate is at least 50 percent and no greater than 100 percent of the mass transfer rate over a period of time;
the mass transfer rate is at least 50 percent and no greater than 100 percent of the feed rate over a concurrent period of time; and
the period of time and the concurrent period of time are the same 10 second period.

7. The method of claim 3, comprising:
feeding the composition into the first chamber of the system at a feed rate, which is the amount of the molecule that is fed into the first chamber per unit time;
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the composition has a surface-area-to-volume ratio that is greater than 500 per meter;
the composition off-gasses the vaporized molecule at a vaporization rate;
the method comprises condensing the vaporized molecule into the condensed molecule from a first portion of the composition in the second chamber and concurrently converting the molecule into the vaporized molecule from a subsequent portion of the composition in the first chamber;
the method is performed such that the collection rate is at least 50 percent and no greater than 100 percent of the mass transfer rate over a period of time;
the mass transfer rate is at least 50 percent and no greater than 100 percent of the feed rate over a concurrent period of time; and
the period of time and the concurrent period of time are the same 10 second period.

8. The method of claim 3, comprising:
feeding the composition into the first chamber of the system at a feed rate, which is the amount of the molecule that is fed into the first chamber per unit time;
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the composition has a surface-area-to-volume ratio that is greater than 500 per meter;
the composition off-gasses the vaporized molecule at a vaporization rate;
the method comprises condensing the vaporized molecule into the condensed molecule from a first portion of the composition in the second chamber and concurrently converting the molecule into the vaporized molecule from a subsequent portion of the composition in the first chamber;
the method is performed such that the collection rate is at least 50 percent and no greater than 100 percent of the mass transfer rate over a period of time; and
the mass transfer rate is at least 50 percent and no greater than 100 percent of the feed rate over a concurrent period of time.

9. The method of claim 3, comprising:
bombarding the composition with a sweep gas;
converting the molecule into the vaporized molecule at a mass transfer rate, which is the amount of the molecule that the composition off-gases minus the amount of the vaporized molecule that recondenses onto the composition per unit time; and
condensing the vaporized molecule into the condensed molecule at a collection rate, which is the amount of the vaporized molecule that is condensed into the condensed molecule per unit time,
wherein:
the bombarding increases the vapor pressure of the molecule;
increasing the vapor pressure of the molecule comprises latent heat transfer from the gas phase to the composition;
the composition has a surface-area-to-volume ratio that is greater than 500 per meter; and
the method comprises condensing the vaporized molecule into the condensed molecule from a first portion of the composition in the second chamber and concurrently converting the molecule into the vaporized molecule from a subsequent portion of the composition in the first chamber.

10. The method of claim 3, wherein:
the composition has a surface-area-to-volume ratio that is greater than 500 per meter; and
the method comprises condensing the vaporized molecule into the condensed molecule from a first portion of the composition in the second chamber and concurrently converting the molecule into the vaporized molecule from a subsequent portion of the composition in the first chamber.

11. The method of claim 3, wherein the composition has a surface-area-to-volume ratio that is greater than 500 per meter.

12. The method of claim 3, comprising:
feeding the composition into the first chamber of the system at a feed rate, which is the amount of the molecule that is fed into the first chamber per unit time;
bombarding the composition with a sweep gas; and
converting the molecule into the vaporized molecule at a mass transfer rate, which is the amount of the molecule that the composition off-gases minus the amount of the vaporized molecule that recondenses onto the composition per unit time,
wherein:
the bombarding increases the vapor pressure of the molecule;
increasing the vapor pressure of the molecule comprises sensible heat transfer from the gas phase to the composition;
the composition has a surface-area-to-volume ratio that is greater than 500 per meter; and
the surface-area-to-volume ratio of the composition results in a mass transfer rate of at least 5 micrograms of the molecule per gram of the composition per second at the temperature and the pressure of the gas phase.

13. The method of claim 3, comprising:
feeding the composition into the first chamber of the system at a feed rate, which is the amount of the molecule that is fed into the first chamber per unit time; and
bombarding the composition with a sweep gas,
wherein:
the bombarding increases the vapor pressure of the molecule;
increasing the vapor pressure of the molecule comprises sensible heat transfer from the gas phase to the composition; and
the composition has a surface-area-to-volume ratio that is greater than 500 per meter.

14. The method of claim 3, wherein:
the composition comprises the accessory molecule at a concentration of no greater than 20 percent by mass;
the method comprises generating sufficient pressure within the biological cells to rupture at least 75 percent of the cell membranes; and
the method comprises vaporizing the accessory molecule and rupturing the cell membranes in a total time of no greater than 60 seconds.

15. The method of claim 3, wherein:
the composition comprises the accessory molecule at a concentration of no greater than 20 percent by mass; and
the method comprises vaporizing the accessory molecule and rupturing the cell membranes in a total time of no greater than 60 seconds.

16. The method of claim 3, wherein:
the composition comprises the accessory molecule at a concentration of no greater than 20 percent by mass; and
the method comprises vaporizing the accessory molecule and rupturing the cell membranes in a total time of no greater than 60 seconds.

17. The method of claim 3, comprising generating sufficient pressure within the biological cells to rupture at least 75 percent of the cell membranes.

18. The method of claim 3, wherein the composition comprises the accessory molecule at a concentration of no greater than 20 percent by mass.

19. The method of claim 3, comprising vaporizing the accessory molecule and rupturing the cell membranes in a total time of no greater than 60 seconds.

20. The method of claim 3, comprising bombarding the composition with a sweep gas.
